(19) United States
(12) Patent Application Publication
(10) Pub. No.: US 2009/0005425 A1
(43) Pub. Date: Jan. 1, 2009

(54) Complex Formulation Comprising Amlodipine Camsylate And Simvastatin and Method For Preparation Thereof

(76) Inventors: Jong Soo Woo, Gyeonggi-do (KR); Moon Hyuk Chi, Gyeonggi-do (KR); Yong II Kim, Gyeonggi-do (KR); Hong Gi Yi, Gyeonggi-do (KR)

Correspondence Address: SUGHRUE MION, PLLC, 2100 PENNSYLVANIA AVENUE, N.W., SUITE 800, WASHINGTON, DC, 20037, US

(21) Appl. No.: 12/159,418

(22) PCT Filed: Dec. 22, 2006
(86) PCT No.: PCT/KR2006/005658

§ 371 Date: Jun. 27, 2008

(30) Foreign Application Priority Data
Dec. 27, 2005 (KR) 10-2005-0130531

(51) Int. Cl. A61K 31/4422

(52) U.S. Cl. 514/356

(57) ABSTRACT

The present invention relates to a complex formulation for oral administration including amlodipine camsylate and simvastatin, and a preparation method thereof. The complex formulation of the present invention comprising amlodipine camsylate, simvastatin and a stabilizing agent can be used advantageously for preventing and treating diseases such as hyperlipidemia, atherosclerosis, hypertension, and cardiovascular disease.

### FIELD OF THE INVENTION

The present invention relates to a complex formulation for oral administration comprising amlodipine camsylate and simvastatin, and a method for preparation thereof.

### BACKGROUND OF THE INVENTION

Hyperlipidemia or serum lipid level elevation is related to the occurrence of cardiovascular diseases and arteriosclerosis. The hyperlipidemia includes hypercholesterolemia, familial dysbetalipoprotenemia, diabetic dyslipemia, nephritic dyslipemia and familial combined hyperlipidemia. Hypercholesterolemia, a representative example of hyperlipidemia, is caused by elevated serum LDL (low-density lipoprotein)-cholesterol and total cholesterol levels, and the treatment of hypercholestrolemia by reducing the serum lipid level, especially the LDL-cholesterol level, makes it possible to lower the risk of cardiovascular disorders, which leads to delayed progression of arteriosclerosis (American diabetes association, Diabetic care, 23 (suppl.), S57-S65, 2000). Therefore, there have been many studies on lipid-lowering therapy for delaying the progression of arteriosclerosis or alleviating arteriosclerosis so as to reduce the risk of cardiovascular disorders, e.g., coronary heart disease, in a patient diagnosed as hyperlipidemia or hypercholestrolemia.

Hypertension is accompanied by hyperlipidemia in many cases, which may cause cardiac disorders such as angina pectoris. Thus, it is very important to control hypertension together with the cholesterol level when a patent is suffering from coronary heart diseases, so that the risk or fatality arising from cardiovascular disorders can be reduced.

For example, Kramsch et al. have disclosed that a calcium channel blocking agent such as amlodipine, an antihypertension agent, can be administered together with a lipid-lowering agent to enhance the therapeutic effects against atherosclerosis (Kramsch et. al., Journal of Human Hypertension, Suppl. 1, 53-59, 1995), and Lichtlen P. R. et al. have reported that an early atherosclerotic disease in human can be effectively treated by co-administering a calcium channel blocking agent (Lichtlen P. R. et al., Lancet, 335, 1109-1139, 1990; and Waters D. et al., Circulation, 82, 1940-1953, 1990).

Further, U.S. Pat. No. 4,681,893 disclosed that some statin drugs including atrovastatin are useful for treating atherosclerosis, and it has been reported that in case of administering a statin drug (pravastatin or lovastatin) together with a calcium channel blocking agent (amlodipine), atherosclerotic diseases can be better treated through synergistic effects of the two drugs (Jukema et. al., Circulation, Suppl. 1, 1-197, 1995; and Orekhov et. al., Cardiovescular Drug and Theraphy, 11, 350, 1997). However, Caduet® (Pfizer), a commercially available atrovastatin-amlodipine besylate complex formulation wherein astrovastatin is a HMG-CoA reductase inhibitor and amlodipine besylate is a therapeutic for hypertension, has the problem that the photostability of amlodipine besylate is poor, implying that amlodipine besylate may be easily degraded during the starage of the complex formulation.

The present inventors have found that a complex formulation for oral administration comprising amlodipine camsylate, which has superior photostability than amlodipine besylate's, exhibits improved stability.

### SUMMARY OF THE INVENTION

Accordingly, it is an object of the present invention to provide a complex formulation comprising amlodipine camsylate and simvastatin, which are therapeutics for hypertension and hyperlipidemia, respectively, and a method for preparation thereof.

In accordance with one aspect of the present invention, there is provided a complex formulation for oral administration comprising amlodipine camsylate, simvastatin, and a stabilizing agent.

### BRIEF DESCRIPTION OF THE DRAWINGS

The above and other objects and features of the present invention will become apparent from the following description of the invention, when taken in conjunction with the accompanying drawings which respectively show:

FIG. 1: a schematic diagram of the inventive complex formulation comprising amlodipine camsylate and simvastatin;

FIG. 2: a graph showing the changes in the amlodipine besylate and amlodipine camsylate contents when exposed to sunlight;

FIG. 3: the amounts of degradation products of amlodipine besylate and amlodipine camsylate when exposed to sunlight;

FIG. 4: the amounts of degradation products of amlodipine besylate and amlodipine camsylate when exposed to incandescent light;

FIG. 5: the changes in the amlodipine content when the solid dispersions prepared in Comparative Example 1 and Examples 1 to 4 were subjected to stability tests;

FIG. 6: the amounts of the degradation products of amlodipine generated when the solid dispersions prepared in Comparative Example 1 and Examples 1 to 4 were subjected to stability tests;

FIG. 7: the change in the amlodipine content during the stability test of the complex formulations prepared in Comparative Example 2 and Examples 5 to 7;

FIG. 8: the amounts of the degradation products of amlodipine during the stability tests of the complex formulations prepared in Comparative Example 2 and Examples 5 to 7;

FIG. 9: the changes in the amlodipine content during the stability tests of the complex formulations prepared in Examples 7 and Comparative example 3;

FIG. 10: the amount of the degradation product of amlodipine during the stability tests for the complex formulations prepared in Examples 7 and Comparative example 3;

FIG. 11: the changes in the simvastatin content during the stability tests of the complex formulations prepared in Comparative Examples 2 and 3, and Examples 5 to 7;

FIG. 12: the changes in the amlodipine content through the comparative stability tests of the complex formulation prepared in Example 7 and a control formulation, Caduet®; and

FIG. 13: the amounts of the degradation products of amlodipine during the comparative stability test for the complex formulation prepared in Example 7 and a control formulation, Caduet®.

### DETAILED DESCRIPTION OF THE INVENTION

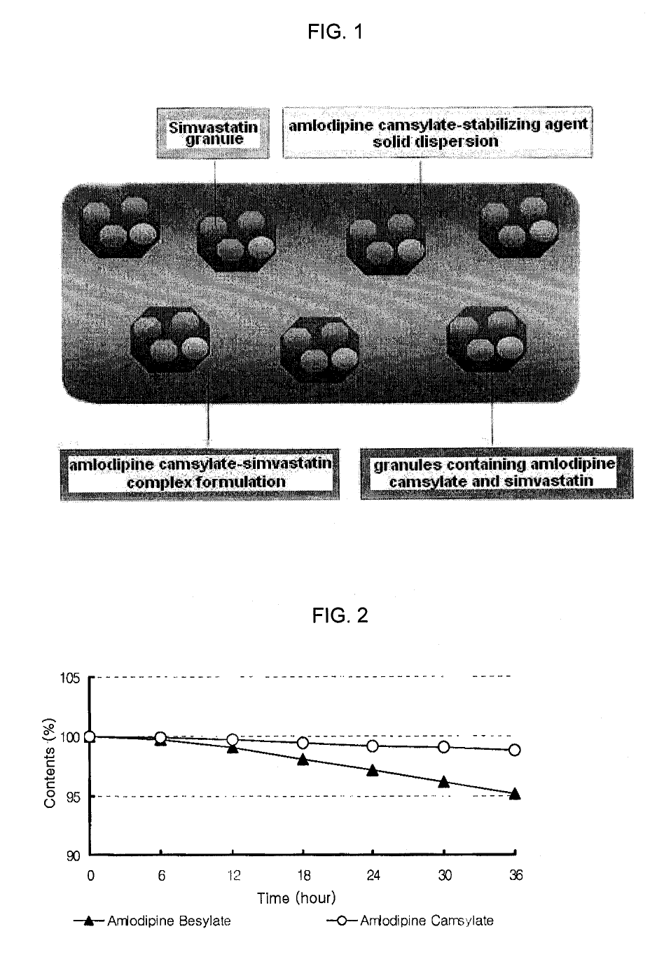

The complex formulation of the present invention is characterized by comprising amlodipine camsylate and simvastatin, which are therapeutics for hypertension and hyperlipidemia, respectively, as shown in FIG. 1.

Each ingredient of the inventive formulation is described in detail as follows:

1) Pharmacologically Active Ingredient

The pharmaceutically active ingredient of the complex formulation according to the present invention comprises amlodipine camsylate, which is a blocking agent for calcium channel, used for treating hypertension; and simvastatin (U.S. Pat. Nos. 4,448,784 and 4,450,171), which is a HMG-CoA reductase inhibitor, used for treating hyperlipidemia and arteriosclerosis by lowering the lipoprotein or lipid level in blood. The amlodipine camsylate has superior photostability than amlodipine besylate known as the most appropriate amlodipine salt so far.

Amlodipine camsylate may be employed in an amount ranging from 0.5 to 20% by weight, preferably from 1 to 10% by weight based on the total weight of the complex formulation. When the amount is less than 0.5% by weight, its therapeutic effect cannot be expected, and when more than 20% by weight, a safety problem may arise because it exceeds the allowable daily dose.

Simvastatin may be employed in an amount ranging from 0.5 to 50% by weight, preferably from 1 to 40% by weight based on the total weight of the complex formulation. When the amount is less than 0.5% by weight, its therapeutic effect cannot be expected, and when more than 50% by weight, a safety problem may arise because it exceeds the allowable daily dose.

2) Stabilizing Agent

The complex formulation according to the present invention comprises a stabilizing agent which prevents the oxidation of amlodipine camsylate and simvastatin used as a pharmaceutically active ingredient.

The stabilizing agent used in the present invention may be any one of the known stabilizing agents, and exemplary stabilizing agents include butylated hydroxy toluene (BHT), butylated hydroxy anisol (BHA), erythorbic acid, ascorbic acid, tocopherol and the like.

In present invention, stabilizing agent may be employed in an amount ranging from 0.001 to 100% by weight, preferably 0.002 to 50% by weight based on the weight of amlodipine camsylate. When the amount is less than 0.001% by weight of amlodipine camsylate, it is difficult to attain the expected drug stability, and when more than the weight of amlodipine camsylate, a safety problem may arise because it exceeds the allowable daily dose.

3) Pharmaceutically Acceptable Additive

The sustained release formulation of the present invention may further comprise at least one of the known pharmaceutically acceptable additives such as a dispersing agent, binder, lubricating agent, sweetening agent, excipient and the like, in order to prepare a solid formulation suitable for oral administration. Representative examples of the pharmaceutically acceptable additive may include any one of the binder generally used in pharmacy, such as polyvinylpyrrolidone (PVP), gelatin, hydroxypropyl cellulose and Copovidone, and any one of the lubricating agent generally used in pharmacy, such as sucrose fatty acid ester, talc, light anhydrous silicic acid, zinc and magnesium salts of stearic acid and the like.

The inventive complex formulation for oral administration comprising said ingredients may be prepared by the following steps:

1) dissolving amlodipine camsylateand a stabilizing agent in an organic solvent to obtain a solution, and removing the organic solvent from the solution to obtain a solid dispersion; and

2) mixing the solid dispersion obtained in step 1 with simvastatin and a pharmaceutically acceptable additive to obtain a mixture, and granulating the mixture by wet milling to obtain granules, followed by formulating the granules.

In step 1), the organic solvent may be methanol, ethanol, dichloromethane, chloroform and the like, and the solid dispersion may be prepared by a conventional method such as spray-drying, solvent evaporating, micropulverizing-wetting, melting, and freeze-drying methods.

In step 2), a solvent such as water, ethanol and dichloromethane may be employed to form a binder solution during the preparation of the granules comprising the pharmaceutically active ingredients of the complex formulation, amlodipine camsylate and simvastatin.

Further, the above method according to the present invention may further comprise the step of coating the obtained complex formulation with a film layer for protecting the formulation from degenerative factors such as light and moisture as well as for enhancing the patient compliance (e.g., by blocking a bitter taste). The outer film layer may be a light-shielding film layer, moisture-proof film layer or sugar film layer.

The preferable film layer may comprises at least one of the known water-soluble film-forming materials such as hydroxypropylmethylcellulose (HPMC), hydroxypropylcellulose (HPC), hydroxyethylcellulose (HEC), celluloseacetatephthalate (CAP), ethylcellulose (EC), methylcellulose (MC), polymethacrylate, Kollicoat® (BASF, Germany) and Opadry® (Colorcon, USA).

The water-soluble film layer may be employed in an amount ranging from 0.5 to 20% by weight, preferably 1 to 10% by weight based on the weight of the inventive complex formulation. When the amount is less than 0.5% by weight, the film becomes unstable, and when more than 20% by weight, it adversely affects the drug release.

In addition, the water-soluble film layer may further comprise plasticizers such as polyethyleneglycol (PEG), glycerol triacetate and acetylated monoglyceride.

The amlodipine camsylate-simvastatin complex formulation of the present invention prepared by the above method has an improved effect of the pharmaceutically active ingredients by releasing them rapidly and has improved stability of amlodipine camsylate and simvastatin by comprising the stabilizing agent. The inventive complex formulation can be effectively used for preventing and treating hyperlipidemia, arteriosclerosis, hypertension, cardiovascular disease and the combined disease thereof when orally administered once per day at a single dose.

The following Examples are intended to further illustrate the present invention without limiting its scope.

### EXAMPLES 1 TO 4 AND COMPARATIVE EXAMPLE 1

### Preparation of Solid Dispersion Comprising Amlodipine Camsylate and a Stabilizing Agent

Amlodipine camsylate, an active ingredient, and BHT (UENO Fine Chemical, USA), a stabilizing agent, were dissolved in 100 ml of a mixture of ethanol and dichloromethane (2:8, w/w) according to the amounts described in Table 1, respectively, and each of the resulting mixture was subjected to spray-drying to obtain a solid dispersion

TABLE 1

| Component | Comparative | Exam- | Exam- | Exam- | Exam- |
|---|---|---|---|---|---|
| (mg/tablet) | Example 1 | ple 1 | ple 2 | ple 3 | ple 4 |
| amlodipine | 7.84 | 7.84 | 7.84 | 7.84 | 7.84 |
| camsylate | | | | | |
| BHT | — | 0.001 | 0.01 | 0.05 | 0.1 |

The conditions for spray-drying procedure:

1) Equipment: Buchi Mini Spray Dryer B-191

2) Temperature: influx: 80° C., effluent: 52° C.

3) Air Flow: 500 NI/h

4) Pump (%): 12% (spraying in amount of about 120 ml per hour)

### EXAMPLES 5 TO 7 AND COMPARATIVE EXAMPLE 2

### Preparation of an Amlodipine Camsylate-Simvastatin Complex Formulation for Oral Administration

Complex formulations for oral administration were prepared using the components described in Table 2. The solid dispersions prepared in Examples 1 to 4 and Comparative Example 1 were each mixed with simvastatin, an active ingredient for treating hyperlipidemia, microcrystalline cellulose, mannitol, dibasic calcium phosphate and sodium starch glycolate. Then, a binder solution prepared by dissolving 3 mg of Povidone (BASF, Germany) in about 50 ml of purified water was added to the mixture, which was granulated by wet milling to obtain granules. The granules were dried and passed through a 750 μm-sieve. Magnesium stearate as a lubricating agent was added to the granules and an amlodipine camsylate-simvastatin complex formulation for oral administration was prepared by a conventional tabletting method.

TABLE 2

| | | Comparative | Exam- | Exam- | Exam- |
|---|---|---|---|---|---|
| Component (mg/tablet) | | Example 2 | ple 5 | ple 6 | ple 7 |
| Part of | amlodipine | 7.84 | 7.84 | 7.84 | 7.84 |
| granules | camsylate | | | | |
| | simvastatin | 20 | 20 | 20 | 20 |
| | BHT | — | 0.1 | 0.2 | 0.5 |
| | micro- | 47 | 47 | 47 | 47 |
| | crystalline | | | | |
| | cellulose | | | | |
| | dibasic | 50 | 50 | 50 | 50 |
| | calcium | | | | |
| | phosphate | | | | |
| | mannitol | 60 | 60 | 60 | 60 |
| | sodium starch | 10 | 10 | 10 | 10 |
| | glycolate | | | | |
| | Povidone | 3 | 3 | 3 | 3 |
| Mixing | magnesium | 2 | 2 | 2 | 2 |
| after | stearate | | | | |
| granulating | | | | | |

### COMPARATIVE EXAMPLE 3

### Preparation of Amlodipine Camsylate-Simvastatin Complex Formulation for Oral Administration

A complex formulation for oral administration was prepared using the components listed in Table 3. Simvastatin, microcrystalline cellulose, mannitol, dibasic calcium phosphate and sodium starch glycolate were mixed together, and a binder solution prepared by dissolving 3 mg of Povidone (BASF, Germany) in about 50 ml of purified water was added thereto. The resulting mixture was granulated by wet milling to obtain granules. The granules were dried and passed through a 750 μm-sieve. The solid dispersion comprising amlodipine camsylate and BHT, prepared by the methods of Examples 1 to 4, was added to the sieved granules. Then, magnesium stearate, a lubricating agent, was added to the resulting mixture, and an amlodipine camsylate-simvastatin complex formulation for oral administration was prepared by a conventional tabletting method.

TABLE 3

| Component (mg/tablet) | | Comparative Example 3 |
|---|---|---|
| Part of granules | simvastatin | 20 |
| | microcrystalline cellulose | 47 |
| | dibasic calcium phosphate | 50 |
| | mannitol | 60 |
| | sodium starch glycolate | 10 |
| | Povidone | 3 |
| Mixing after | amlodipine camsylate | 7.82 |
| granulating | BHT | 0.5 |
| | magnesium stearate | 2 |

### REFERENCE EXAMPLE

### The Comparative Test for the Stability of Amlodipine Besylate and Amlodipine Camsylate

Amlodipine camsylate and amlodipine besylate was exposed to sunlight or an incandescent light (220 V, 100 W), at 40° C. under 75% relative humidity. The change in the amlodipine content and the amount of the degradation product of amlodipine, the compound of formula (I), was measured under the conditions described in Table 4. The results are shown in FIGS. 2 to 4.

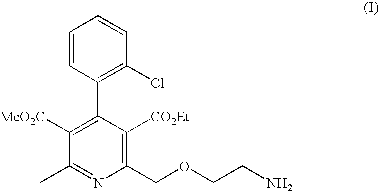

TABLE 4

| | Conditions for quantitative | Conditions for analysis of | | |
|---|---|---|---|---|
| | analysis | degradation products | | |
| Detector | Absorption spectrometer for | Absorption spectrometer for | | |
| | ultraviolet part(237 nm) | ultraviolet part(237 nm) | | |
| Column | A column packed with 5 μm of | A column packed with 3.5 μm of | | |
| | octadecylsilylated silica gel in | octylsilylated silica gel in a stainless | | |
| | a stainless tube (4.6 mm × 15 | tube (4.6 mm × 10 mm) | | |
| | mm) | | | |
| Temperature | 25° C. | 45° C. | | |
| of column | | | | |
| Mobile phase | Methanol/0.03M potassium | 0 min: | A 100% | B 0% |
| | phosphate monobasic | 14 min: | A 35% | B 65% |
| | solution = 60/40 | 21 min: | A 0% | B 100% |
| | | 39 min: | A 100% | B 0% |

| | | |
|---|---|---|
| | | *A (mobile phase A) - 0.05M |
| | | perchloric acid buffer solution (Ph |
| | | 2.75):acetonitrile = 65:35 |
| | | *B (mobile phase B) - 0.05M |
| | | perchloric acid buffer solution (Ph |
| | | 2.75):acetonitrile = 35:65 |
| Flow rate | 1.5 ml/min | 1.0 ml/min |
| Injection | 20 μl | 20 μl |
| volumn | | |
| Preprocessing | Shaking 5 mg of amlodipine | Dissolving about 32 mg of |
| for samples | gained from a sample for 30 | amlodipine gained from a sample in |
| | min in 100 ml of mobile phase | a solution of 0.02 M of acetate buffer |
| | and filtration in 200 ml of | solution (pH 5.0):acetonitrile = 1:1 |
| | mobile phase | and filtration |

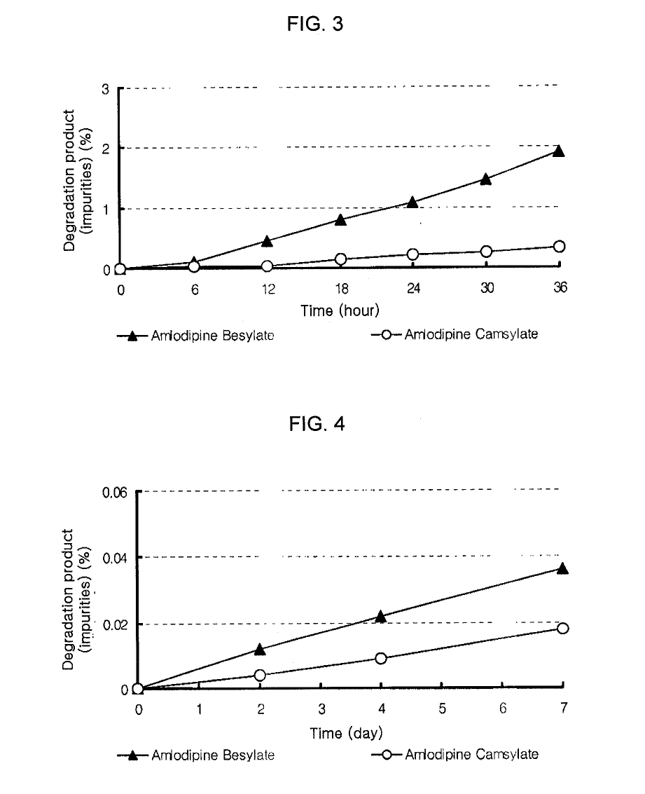

FIG. 2 shows the time-dependant degradation rate of amlodipine besylate and amlodipine camsylate by the action of sunlight; FIG. 3 shows the amount of rate of impurity generation from amlodipine by the action of sunlight; and FIG. 4, the rate of impurity generation from amlodipine caused by incandescent light exposure. The above results imply that amlodipine camsylate has superior photostability as compared with amlodipine besylate.

### TEST EXAMPLE 1

### Stability Test of a Solid Dispersion of Amlodipine Camsylate-Stabilizing Agent

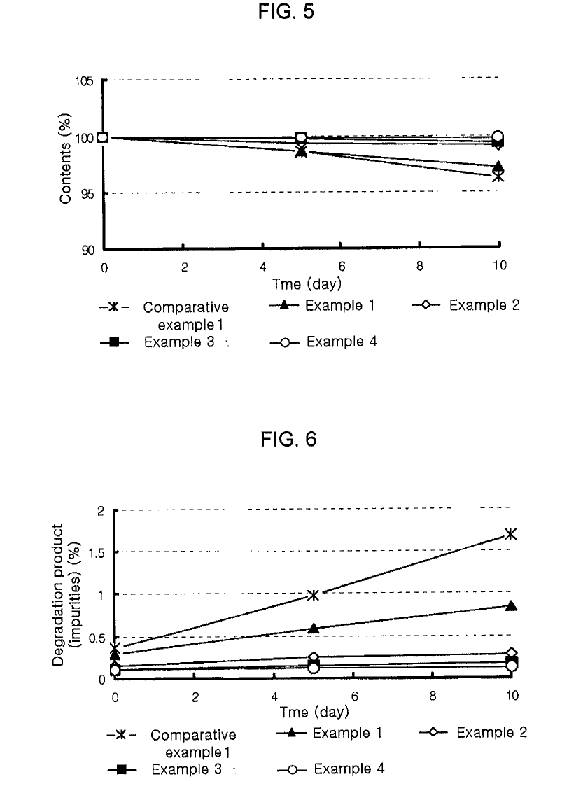

The solid dispersions of Comparative Example 1 and Examples 1 to 3 were each exposed to sunlight or incandescent light (220 V, 100 W), and the changes in the amlodipine content and the compound of formula (I), a degradation product of amlodipine, were analyzed under the conditions described in Table 4. The results are shown in FIGS. 5 and 6.

As shown in FIGS. 5 and 6, the stability of amlodipine improves as the amount of BHT, a stabilizing agent, increases.

### TEST EXAMPLE 2

### Stability Test of a Solid Dispersion of Amlodipine Camsylate-Simvastatin

The complex formulations of Comparative Examples 2 and 3, and Examples 5 to 7 were each placed in an HDPE bottle containing about 5 g of silica gel and the changes in the contents of simvastatin, amlodipine, and the degradation product of amlodipine (impurities of formula (I)) were analyzed at 60° C. under 75% relative humidity. The amounts of amlodipine and the degradation product were determined by the method of Test Example 1 and the simvastatin content was analyzed according to the method described under item “simvastatin tablets” in U.S. pharmacopoeia (28^{th} amendment).

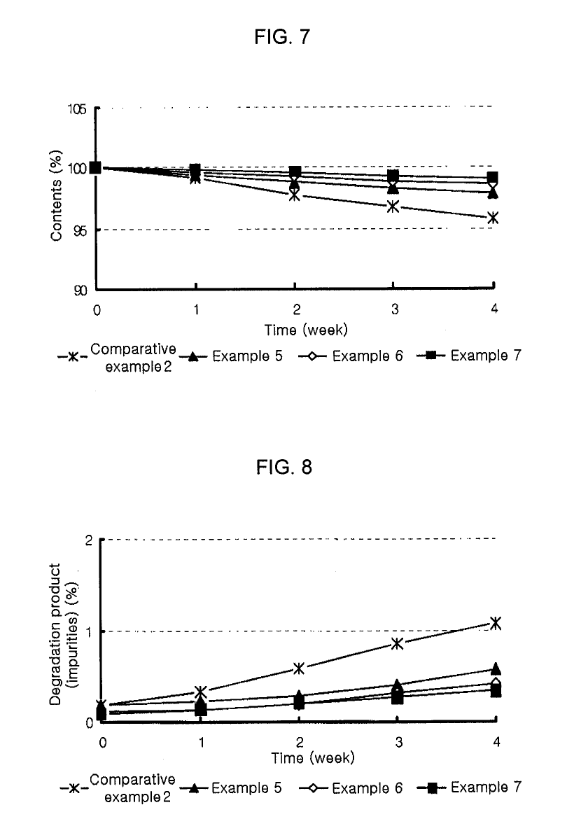

FIG. 7 and FIG. 8 show the changes in the contents of amlodipine and its degradation product, respectively, showing that the stability of amlodipine camsylate increases with the amount of BHT, the stabilizing agent.

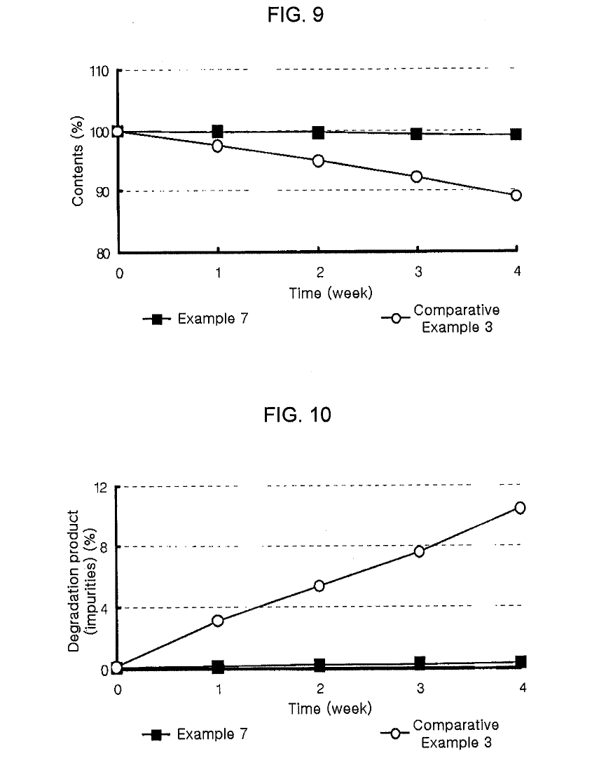

Further, as can be seen in FIGS. 9 and 10, the stability of amlodipine is poor when a granule containing simvastatin was prepared first and then amlodipine camsylate was mixed with the granule (Comparative Example 3). This is because the increased probability for amlodipine camsylate to contact directly with magnesium stearate, the lubricating agent, affects its stability. On the other hand, the stability of amlodipine is satisfactory when amlodipine camsylate and simvastatin are subjected to granulation together as described in Example 7, for the decreased chance for amlodipine camsylate to contact magnesium stearate.

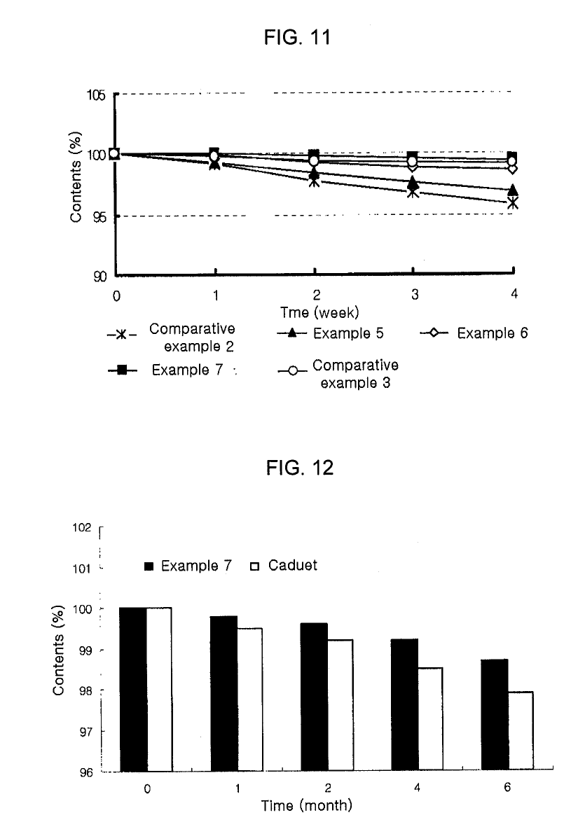

FIG. 11 shows the change in the simvastatin content, showing that BHT elevates the stability of simvastatin.

### TEST EXAMPLE 3

### Comparative Stability Test of a Complex Formulation as Compared with a Control Formulation

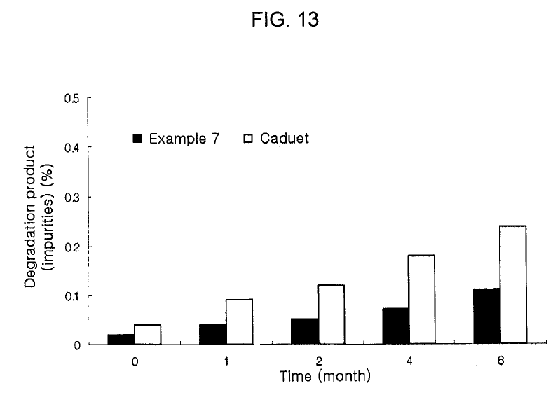

The amlodipine camsylate-simvastatin complex formulation of Example 7 and the amlodipine besylate-atorvastatin complex formulation, Caduet® (Pfizer), which is sold at a market currently in U.S.A., were each placed in a HDPE bottle containing about 5 g of silica gel and stored at 40° C. under 75% relative humidity for 6 months. Then, the changes in the contents of amlodipine and its degradation product were analyzed by the method of Test Example 2. The results are shown in FIGS. 12 and 13.

As can be seen in FIGS. 12 and 13, the stability of amlodipine in the complex formulation of the present invention was greatly improved as compared with the control formulation.

While the invention has been described with respect to the above specific embodiments, it should be recognized that various modifications and changes may be made to the invention by those skilled in the art which also fall within the scope of the invention as defined by the appended claims.

What is claimed is:

1. A complex formulation for oral administration comprising amlodipine camsylate, simvastatin and a stabilizing agent.

2. The complex formulation of claim 1, wherein the amount of amlodipine camsylate ranges from 0.5 to 20% by weight based on the weight of the complex formulation.

3. The complex formulation of claim 1, wherein the amount of the simvastatin ranges from 0.5 to 50% by weight based on the weight of the complex formulation.

4. The complex formulation of claim 1, wherein the stabilizing agent is selected from the group consisting of butylated hydroxy toluene (BHT), butylated hydroxy anisol (BHA), erythorbic acid, ascorbic acid, tocopherol, and a mixture thereof.

5. The complex formulation of claim 1, wherein the amount of the stabilizing agent ranges from 0.001 to 100% by weight based on the weight of amlodipine camsylate.

6. The complex formulation of claim 1, which further comprises a pharmaceutically acceptable additive selected from the group consisting of microcrystalline cellulose, dibasic calcium phosphate, sodium starch glycolate, magnesium stearate and a mixture thereof.

7. A process for preparing the complex formulation of claim 1, which comprises:
1) dissolving amlodipine camsylate and the stabilizing agent in an organic solvent, and removing the organic solvent from the resulting solution to obtain a solid dispersion; and
2) mixing the solid dispersion obtained in step 1 with simvastatin and a pharmaceutically acceptable additive to obtain a mixture, and granulating the mixture by wet milling to obtain granules, followed by formulating the granules.

8. The method of claim 7, wherein the removal of the organic solvent in step 1) is carried out by spray-drying, solvent evaporating, micropulverizing-wetting, melting or freeze-drying methods.

9. The method of claim 7, which further comprises the step of coating the outer surface of the complex formulation with a film layer.

10. The method of claim 9, wherein the film layer is made of a water-soluble material selected from the group consisting of hydroxypropylmethyl cellulose (HPMC), hydroxypropyl cellulose (HPC), hydroxyethyl cellulose (HEC), celluloseacetate phthalate (CAP), ethyl cellulose (EC), methyl cellulose (MC), polymethacrylate, Kollicoat® (BASF, Germany) and Opadry® (Colorcon, USA).

11. The method of claim 9, wherein the film layer is a light-shielding film layer, a moisture-proof film layer, or a sugar film layer.

12. The method of claim 9, wherein the amount of the film layer ranges from 0.5 to 20% by weight based on the weight of the complex formulation.
